(19) United States
(12) Patent Application Publication
(10) Pub. No.: US 2014/0303483 A1
(43) Pub. Date: Oct. 9, 2014

(54) BREAST BIOPSY SYSTEM USING MR AND GAMMA IMAGING

(71) Applicants: James Schellenberg, Winnipeg (CA); Cheryl Dika, Winnipeg (CA); Erin Riediger, Winnipeg (CA)

(72) Inventors: James Schellenberg, Winnipeg (CA); Cheryl Dika, Winnipeg (CA); Erin Riediger, Winnipeg (CA)

(73) Assignee: CUBRESA INC., Winnipeg, MB (CA)

(21) Appl. No.: 14/128,112

(22) PCT Filed: Jun. 26, 2012
(86) PCT No.: PCT/CA2012/050423

§ 371 Date: Apr. 7, 2014

Related U.S. Application Data
(60) Provisional application No. 61/500,421, filed on Jun. 23, 2011.
(60) Provisional application No. 61/500,421, filed on Jun. 23, 2011.

(51) Int. Cl. A61B 6/12; A61B 10/02; A61B 5/00; A61B 6/00; A61B 5/055; G01R 33/48

(52) U.S. Cl. CPC A61B 6/12; A61B 5/0555; G01R 33/4808; A61B 5/708; A61B 6/4258; A61B 10/0233; A61B 6/481; A61B 6/502; A61B 6/4266
USPC 600/411

(57) ABSTRACT

Described herein is the use of gamma cameras in the fringe field of the MRI system. Specifically, an MR image of the breast with lesion identification is first produced. Then, a gamma camera is attached to the existing breast immobilization system for generating one or more gamma images of the breast. The gamma camera is then removed from the breast immobilization system, and a breast biopsy is performed. The gamma camera can then be used to image the biopsy cores that have been removed from the patient in order to verify that the biopsy cores are radioactive, that the biopsy cores extend from one end of the lesion to the other and have a radioactive profile in which the tip is not as radioactive as the middle, and in which the ratio of amount of radioactivity in the middle of the core to the amount of radioactivity that is present in the tip of the core can be expressed as a ratio.

### PRIOR APPLICATION INFORMATION

This application claims the benefit of U.S. Provisional Patent Application 61/500,421, filed Jun. 23, 2011.

### BACKGROUND OF THE INVENTION

MR Breast Biopsy can be characterized as a 1 to 2 hour procedure, in which typically 1, 2, or 3 lesions may be biopsied [1,2,3]. Each biopsy procedure requires approximately 4-5 insertions of the patient into the MRI system. The first insertion is to obtain the starting image; the second insertion is to confirm the guide position; the third insertion is to confirm the needle position prior to insertion into the lesion; and the fourth insertion is often required as a result of patient movement or other reasons. For example, 30% of the time, the guide is placed in the wrong position, and the guide must be moved and the image repeated. If a second and/or third lesion is/are sampled, additional MRI insertions are required. For each lesion site, there may be a need for multiple needle insertions, with between 6 and 12 being possible depending on the needle and biopsy technologies being used. One of the reasons for the large number of needle insertions is because it is difficult to be certain that one is sampling from the lesion Accordingly, it would be useful to have a real-time imaging technology that can provide immediate feedback to confirm that the lesion is being sampled. This is available in ultrasound biopsy, in which the ultrasound transducer is used at the same time as the needle sampling occurs, however there are no real-time MRI biopsy systems currently available. This is because to make real-time MRI biopsy guidance available requires the patient to be in the middle of the bore in imaging position, and this is proving difficult to do both from the engineering and product design perspective as well as from the patient comfort perspective.

Radionuclide based breast biopsy has previously been considered by several authors [5-9]. A stereotactic breast biopsy system using gamma guidance has previously been considered by Welch et al [4].

One of the approaches to improve breast biopsy is to employ several imaging technologies in a hybrid imaging configuration. A hybrid system using PEM and X-ray systems was discussed by Wienberg et al [9]. Previous patents in this area include U.S. Pat. No. 7,711,409 by Keppel et al, and U.S. Pat. No. 6,389,098 by Keppel et al.

U.S. Pat. No. 7,711,409 discusses a co-registration approach to breast biopsy using X-ray and gamma imaging. The inventors discuss using gamma cameras at various angles to the breast in question and opposed gamma cameras.

As well, a previous submission related to MR Gamma Hybrid imaging systems is from Schellenberg, WIPO Patent Application WO/2011/097726.

However, no one has discussed equipment configurations for optimizing breast biopsy using combined MR and Gamma Imaging. Several product architectures are possible, but it is important to note that MR breast biopsy today occurs in the fringe field of the MRI, not in the bore, and so it would be useful to have a gamma system design that can operate in the fringe field.

### SUMMARY OF THE INVENTION

According to a first aspect of the invention, there is provided a method of performing a biopsy comprising:

immobilizing a breast of a patient in need of a biopsy with a breast immobilization apparatus;

inserting the patient into a bore of a magnetic resonance imaging (MRI) device;

taking an MRI image of the breast;

applying a suitable radioisotope for gamma imaging to the patient;

identifying suspicious lesion(s) of interest and their location(s) using the MRI image;

attaching a gamma camera to the breast immobilization apparatus;

visualizing the lesions of interest using the gamma camera; and

performing a biopsy on the selected lesion of interest.

The biopsy may be performed with a gamma-visible needle.

The lesion may be imaged during the biopsy with the gamma camera.

The gamma camera may be used to confirm that biopsy cores are radioactive following the biopsy.

The gamma camera may be used to confirm that biopsy cores extend from one end of the lesion to the other following the biopsy.

The gamma camera may be used to confirm that biopsy cores have a radioactive profile in which the tip is not as radioactive as the middle, and in which the ratio of amount of radioactivity in the middle of the core to the amount of radioactivity that is present in the tip of the core can be expressed as a ratio.

There may be two or more gamma cameras.

The gamma cameras may comprise a first gamma camera having a first scintillator suitable for a first radioisotope and a second gamma camera having a second scintillator suitable for a second radioisotope.

The gamma cameras may comprise a first gamma camera having a first collimator focus length and a second gamma camera having a second collimator focus length.

### BRIEF DESCRIPTION OF THE DRAWINGS

FIG. 1 shows a room view of an MR Room with Gamma Camera

FIG. 2 is a rendering of the gamma camera being used with a monitor and shield.

FIG. 3 showsa small gamma camera head.

FIG. 4 shows a small gamma camera head attached to the square grid attachment and square grid.

FIG. 5 shows a small gamma camera head with rails on the side for retrofit mounting.

FIG. 6 shows a side drawing of a Breast Biopsy Configuration using a small gamma camera on the same side as the biopsy system

FIG. 7 shows a configuration with small gamma camera in front, monitor on the left side, full size camera in rear.

FIG. 8 shows a full camera in the rear, a smaller camera in the front, and a biopsy needle system inserting into the lesion. The needle is gamma visible.

FIG. 9 shows three small cameras with full camera in rear, monitor on the side. The full camera may use parallel collimation, and the small cameras focus in on specific lesion areas.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

For MRI Breast Biopsy using a MR Safe Gamma Camera, the equipment requirements include:
an MRI system with software and hardware
a patient rest and breast immobilization system
a biopsy needle system
a software capability for needle positioning and guidance
a method of gadolinium and radioisotope injection into the patient, and
an MR Safe gamma camera system consisting of:
   one or more gamma camera heads which are MR safe
   an interface module which allows connection between the gamma camera head and the processing system, and which may be MR safe, and
   a processing system consisting of hardware and software, with the processing system typically being outside the mr room and in the MR control room.

Described herein is the use of gamma cameras in the fringe field of the MRI system. Specifically, an MR image of the breast with lesion identification is first produced. Then, a gamma camera is attached to the existing breast immobilization system for generating one or more gamma images of the breast. The gamma camera is then removed from the breast immobilization system, and a breast biopsy is performed. The breast biopsy systems sample the lesion volume by taking a round column of tissue, for example, a column which is 4 mm in diameter and 10 mm in length, and these columns are called a biopsy core. The gamma camera is then used to image the biopsy cores that have been removed from the patient in order to verify that the biopsy cores are radioactive, and that the biopsy cores extend from one end of the lesion to the other and have a radioactive profile in which the tip is not as radioactive as the middle, and in which the ratio of amount of radioactivity in the middle of the core to the amount of radioactivity that is present in the tip of the core can be expressed as a ratio. The ratio of radioactivity from middle to end is important because it has been related to the extent of malignancy that exists within the lesion. For example, if the two ends of the biopsy core are normal tissue, they will have a normal level of radioisotope uptake. If the middle of the core is only 1.5 or 2 times as radioactive, this would generally imply a fairly light level of uptake and a lesion which is not very malignant. However, if the uptake in the middle of the lesion is 5 to 10 times higher than the ends, this implies a strong uptake indicating a strong malignancy in the lesion. Of course, if the middle of the sample has the same radioactivity level as the ends, it implies that the whole core may be taken from normal tissue, which implies that the lesion has been missed.

In some embodiments, the system includes a camera block with rails. A camera block is a square insertion that can be pushed into the square holes of the breast paddle, and which includes a rail extending from the camera block to allow the gamma camera to be mounted onto the rail. A camera block is used beside the needle block and will allow for easy positioning of the small gamma camera. The camera block may have a Mammatome-style rail emitting from the side, such that the rail allows the gamma camera to be pushed onto the camera block. Different camera blocks may have different rail angles, such that if the lesion is 1, 2, 3 cm or some other distance from the surface of the skin, the different rails allow for easy and effective imaging of the lesion while the biopsy is taking place. In other embodiments, the system includes right and left side camera blocks, and the gamma camera has rail guides on both side of its body (right and left) to allow for same ease of positioning and placement.

In other embodiments, the camera block can also be made with a direct, straight-ahead design that allows the gamma camera to be pushed directly into the fenestration square behind which is the lesion of interest.

In some embodiments, there is provided a monitor in the fringe field that is driven by the processing end. In these embodiments, the image and signal processing and user interface calculations can be done by the processing system located in the MRI control room or located elsewhere, and then the results can be passed to the monitor for display. This approach allows the digital equipment to be remotely located from the biopsy site, which eases the electronic noise levels and the amount of equipment in the room. Various remote monitor standards could be used for driving a remote display. Some standards are designed to minimize the amount of data required to drive the display, while other standards could use high and constant data rate approaches or analog approaches. If the processing system is located in the MR control room, then the interface module may be used to convert the signals from a fiber passed physical layer to a cable based physical layer. A monitor may be positioned beside the biopsy area to allow for easy usage by the radiologist. The monitor could be positioned on the wall, on a boom, or in another position; however, in some cases it will be useful to have the monitor positioned in the fringe field of the MRI and close to the breast under intervention.

Preferably, the small gamma camera can be pointed directly back towards the guide, thereby aligning both the lesion (emitting at Tc99m energies of 140 keV) and the fiducialed guide and needle apparatus (emitting at some other energy such as 122 keV for Cobalt 57). The use of the second isotope with a different isotope energy has the main advantage of potentially allowing for embodiments of the invention using a more flexible dual camera system implementation. This second energy may allow for an optimized collimator and/or scintillator to be used to detect the second energy, which may reduce cost or size requirements. In addition, if any dynamic imaging is required of the radioisotope injection trail, it is expected to be easier to employ a two camera approach in which one camera is optimally designed for the first isotope while the other is more optimally designed for the second isotope. As will be appreciated by one of skill in the art, this may include the use of software routines for event detection in which the energy windowing algorithms that are used are different for the two isotopes. Another advantage of the two isotope method is that one camera can be pointed in the direction most optimal for the first isotope, while the second camera can be pointed in the direction that is most optimal for the second isotope. For example, if the two isotopes were the same, then the breast lesion may look like a noise source for the equipment isotope, and the equipment isotope may look like a noise source for the breast lesion equipment. It is also known that the Tc99 isotope will also be taken up in the heart and other organs, and so if a different equipment isotope is used, the heart and other organs will not cause as much noise for the equipment positioning function as they might otherwise cause. As well, because the heart and other organs are Tc99 noise sources, if Tc99 was used for the equipment isotope there might be a restriction in the directions which the equipment tracking cameras could adopt. Instead, in embodiments wherein a second isotope is used, the equipment tracking cameras have more flexibility in positioning.

In some embodiments, more than one, for example, 2 or 3 small gamma cameras are used at the same time, wherein each camera is assessing the uptake level of the various lesions, as discussed above. This can be done for reasons of time or accuracy. For example, if only one small gamma camera is used but there are 3 lesions, then the single camera would need to first image one lesion, then move to image the second lesion, and then be moved to image the third lesion. If there are 3 small gamma cameras, they can each image one of the lesions at the same time, saving time in the procedure. As will be apparent to one of skill in the art, the trade-off for saving time is the cost associated for the hospital or clinic in owning 3 gamma cameras instead of only 1.

In some embodiments, there are a plurality of gamma cameras wherein each gamma camera has a different collimator focal length. For example, one collimator may be a parallel hole collimator that provides a 1 to 1 picture of the breast tissue within it's volume of interest, and another collimator may be a 4:1 microscopic imaging collimator that provides a view of increased detail of its area of interest. These two cameras may point at the same volume at the same time, and be used to provide two views of the same lesion.

In other embodiments, there are 2 or more small gamma cameras, with each gamma camera having a different scintillator. This can be useful if one camera is following the progress of one radioisotope and the other is following a different radioisotope. In this case, the two or more gamma cameras are co-registered using the mechanical registration of the breast immobilization system. Mechanical registration is always preferred to software co-registration because the mechanical registration is typically faster to process, cheaper to implement in design, and is highly accurate compared to software registration. This type of co-registration process can be used in retrofit mode for the many square grid breast immobilization systems that exist in the market.

In an embodiment of the invention, there is provided a method of performing a biopsy comprising: immobilizing a breast of a patient in need of a biopsy with a breast immobilization apparatus; inserting the patient into a bore of a magnetic resonance imaging (MRI) device; taking an MRI image of the breast; applying a suitable radioisotope for gamma imaging to the patient; identifying suspicious lesion(s) and their location(s) using the MRI image; attaching a gamma camera to the breast immobilization apparatus; visualizing the lesions of interest using the gamma camera; and performing a biopsy on the selected lesion.

The biopsy may be performed with a gamma-visible needle. As discussed above, in these embodiments, real-time visualization of the biopsy procedure is possible.

The gamma camera may be used to confirm that biopsy cores are radioactive following the biopsy or to determine the radioactive profile of the biopsy cores.

For example, the gamma camera may be used to confirm that biopsy cores extend from one end of the lesion to the other following the biopsy or to confirm that biopsy cores have a radioactive profile in which the tip is not as radioactive as the middle, and in which the ratio of amount of radioactivity in the middle of the core to the amount of radioactivity that is present in the tip of the core can be expressed as a ratio. As discussed above.

In some embodiments, as discussed above, the system may include two or more gamma cameras. These gamma cameras may be a first gamma camera having a first scintillator suitable for a first radioisotope and a second gamma camera having a second scintillator suitable for a second radioisotope. Alternatively, the gamma cameras may have scintillators suitable for the same isotope and may be used to provide images of one lesion from different angles so as to provide different views of the same lesion or may be arranged such that each camera images one specific lesion.

In other embodiments, the gamma cameras comprise a first gamma camera having a first collimator focus length and a second gamma camera having a second collimator focus length. In this manner, a less detailed image and a more detailed image of a lesion may be generated, as discussed above.

As discussed herein, the imaging discussed above, particularly the gamma imaging, may take place within the fringe field of an MRI device.

In addition, it is of note that in embodiments wherein there are multiple gamma cameras, there may be cameras with different scintillators and/or different collimator focus lengths.

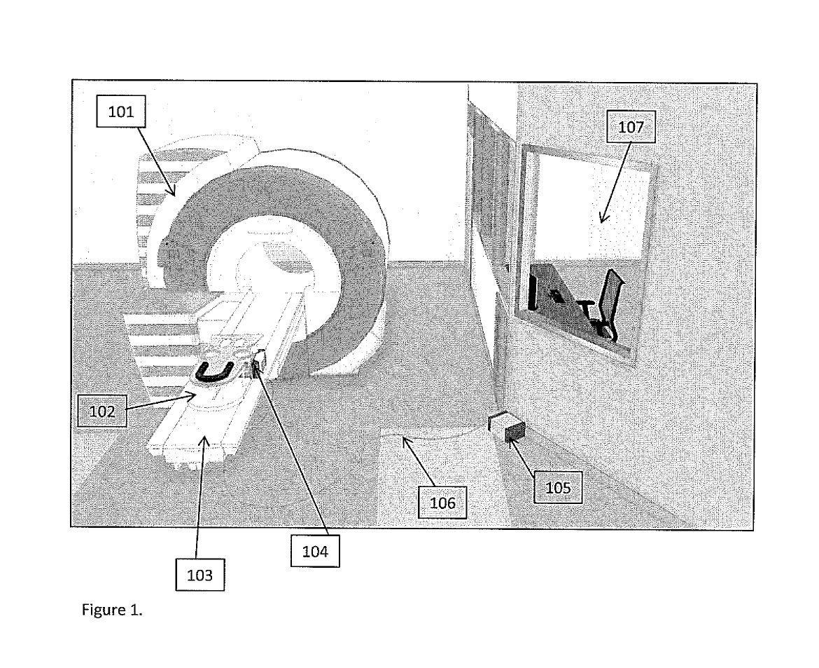

FIG. 1 shows a room view of the MR room with gamma camera. The MR Safe Gamma Camera Head 104 is attached to the Patient Rest System 102. The Patient Rest System is attached to the MRI Table 103, and the imaging of the patient is performed within the MRI magnet 101. The Gamma Camera Head 104 is attached via a cable 106 to an Interface Module 105, which provides various services such as analog to digital conversion, local troubleshooting interface, conversion of cable to a fiber connection, and possible router or network functions. The interface module also provides powering for the gamma camera head in this configuration. The interface module is connected to the MR control room with Processing System 107. As will be appreciated by one of skill in the art, this figure only shows some of the equipment elements that are required for imaging a patient, and the patient and attending staff are also not shown.

For MRI systems today, various configurations are available including 1.5T and 3T magnet strengths and 60 cm and 70 cm bore sizes. For these systems, the MRI table may be fixed or detachable. In the case of a fixed table, the Gamma imaging and biopsy improvement can occur in the fringe field of the MRI. In the case of the detachable table, there is an opportunity to perform MRI imaging and then to roll the patient to another room where gamma imaging can be done.

Three basic workflows are possible with this equipment:

Workflow 1 uses the gamma imaging to assist with the MRI biopsy

Workflow 2 uses the gamma imaging to achieve real-time gamma biopsy in the fringe field of the MRI

Workflow 3 uses the MRI with a detachable table to achieve real-time gamma biopsy in a room adjacent to the MRI room.

The gamma camera or cameras can be attached to a variety of existing breast immobilization equipment for example, those known in the art, such as the square grid immobilization system from NORAS, a version of which is used by Siemens, Hologic, Sentinelle and Invivo. The square grid limits the biopsy needle angle to the horizontal. In addition, there is a post and pillar breast biopsy system which uses vertical and horizontal rods to hold the breast. These vertical and horizontal rods allow larger spaces for the biopsy needle to go through, and the post and pillar is therefore designed to allow the needle to go down at an angle or up at an angle into the breast.

A typical workflow 1 using these elements is:

Image the patient breast using MRI;

Apply the radioisotope, for example, Tc99 which is the FDA approved emitter, into the patient while the patient is still in the bore of the MRI;

Identify the suspicious lesion(s) and their location(s) using the MRI images

Attach the small gamma camera head to the existing breast immobilization system so that the lesion(s) can be visualized using gamma

Identify which lesions if any need to be biopsied

Attach the standard MRI biopsy needle system

Proceed with the biopsy

Optionally, it may be possible in some cases to analyze the biopsy cores for their level of radioactivity, which allows biopsy core sampling to be verified. Evaluation of the biopsy cores immediately upon biopsy allows for immediate feedback to the radiologist, which provides information such as whether the lesion has been sampled and whether the needle is too far to the right or left, or whether the needle passed completely through the lesion or not completely through.

One reason that these two views of a breast lesion are useful is due to the different ways that MRI and gamma imaging work—MRI is anatomical imaging and gamma is functional imaging. For example, in the case of breast lesions, angiogenesis, which is the increase in vasculature that occurs early in the cancer growth, may be visible more easily using gamma techniques than MRI techniques.

A typical Workflow 2 using these elements is:

Image the patient breast using MRI;

Apply the radioisotope into the patient while the patient is still in the bore of the MRI;

Identify the suspicious lesion(s) and their location(s) using the MRI images

Attach the small gamma camera biopsy system to the existing apparatus so that the lesion(s) can be visualized using gamma

Identify which lesions if any need to be biopsied

Attach an MR safe biopsy needle system that is also visible to the gamma camera system

Proceed with a real-time biopsy using gamma imaging as the guidance technology, with the assumption that the guide and/or needle are labelled using radioisotopes;

Optionally, it may be possible in some cases to analyze the biopsy cores for their level of radioactivity, which allows biopsy core sampling to be verified, as discussed above.

A typical Workflow 3 using these elements is:

Image the patient breast using MRI;

Apply the radioisotope into the patient while the patient is still in the bore of the MRI;

Identify the suspicious lesion(s) and their location(s) using the MRI images

Attach the small gamma camera biopsy system to the existing apparatus so that the lesion(s) can be visualized using gamma

Identify which lesions if any need to be biopsied, and verify that the lesion of interest can be imaged using gamma

Move the patient to an adjacent room suitable for real-time gamma biopsy. This movement to a different room is an important trend now occurring in MRI systems. The introduction of a detachable table into the MRI environment now means that a patient can be prepped in one room, then moved into the MRI room for a given procedure or image, and then moved out of the room for those parts of the procedure which do not require further use of the MRI system. In the case of MR Biopsy, if gamma guidance can be introduced after the first MRI image is taken, this implies that time savings of 20 minutes to 80 minutes might be possible depending on the number of lesions that are being sampled. MRI room time is some of the most expensive time in the hospital, and so more flexibility in the scheduling of the room is useful.

Attach a modified biopsy needle system that is visible to the gamma camera system

Proceed with a real-time biopsy using gamma imaging as the guidance technology

Optionally, it may be possible in some cases to analyze the biopsy cores for their level of radioactivity, which allows biopsy core sampling to be verified, as discussed above.

In each case, slightly different equipment items of each type are needed. In workflow 1 a standard MRI biopsy can be done—the gamma technology is used as a method to increase the amount of information for the radiologist. In this workflow, there are probably 4 or 5 insertions into the MRI in this procedure, which is equivalent to the amount of insertions used today. In this workflow, the gamma camera system only needs to operate at a single energy level, such as 140 keV, because the gamma camera system is not being used for real-time guidance,—it is only being used to give additional information on the lesion size, location, and malignancy.

In workflow 2, a real-time biopsy capability is achieved and the number of MRI insertions is reduced. Instead of needing to use the MRI system to check the guide position and the needle position, it is possible to check these positions using the gamma system. This is good for the patient, because typically these MRI sessions are difficult for them. In this case, a second energy level may be presented by the equipment, and so the gamma camera needs to acquire and differentiate both energy levels.

In workflow 3, the detachable table can be used to minimize the time in the MRI suite, thereby attempting to save as much cost as possible. As well, it may be possible to use a gamma camera that is not MR safe, because it may be possible to image the lesion(s) after the patient has been removed from the MRI room. It is still useful in all cases to use a small gamma camera which attaches to the existing breast paddle system. As will be apparent to one of skill in the art, the gamma camera system, once it is attached to the breast immobilization system, can move relative to that system and still will maintain co-registration accuracy.

In all cases, these designs use a retrofit approach in which the gamma camera systems attach to existing breast paddle or patient rest systems, and use the breast paddle and patient rest systems to improve co-registration accuracy.

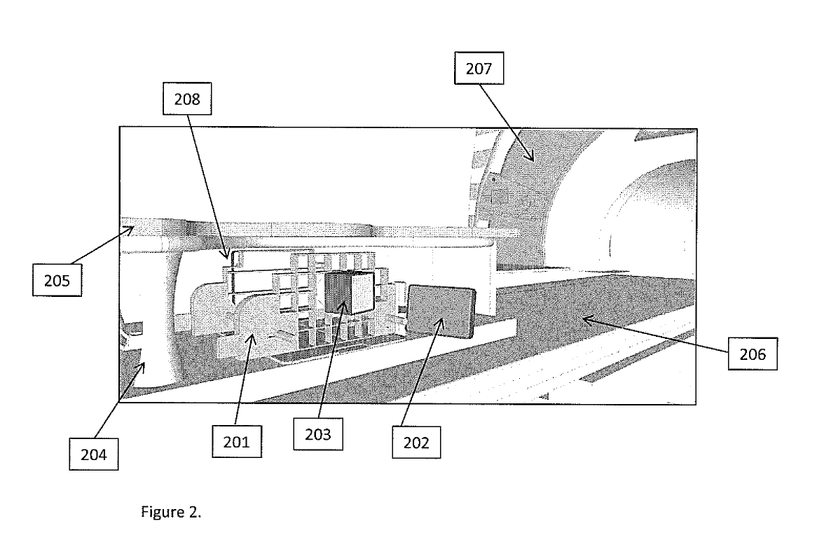

In FIG. 2 is shown a detail of a patient rest system 204 lying on an MRI table 206, in front of a MRI Magnet 207. The system uses a square grid breast immobilization system 201, and attached to the immobilization system is a small gamma camera head 203 and a monitor 202 is attached to the patient rest system. There is upper padding 205 for the patient to lie on in the prone position, with one breast pendant and held between the breast immobilization system. The breast immobilization system has both a front paddle 201 and a rear paddle 208. Attached to the rear paddle is a gamma shield 209 in this case, although a shield is not required in all circumstances. Suitable patient rest systems and breast immobilization systems are made by several vendors, including NORAS MRI products of Wurzburg, Germany.

The monitor is MR Safe, and the information presented on the monitor may include status, diagnostics, powering, and images. For all of these cameras, they may be capable of receiving multiple energy levels, such as 140 keV and then a second energy level as discussed above, or they may be designed for a single energy level.

The NORAS equipment procedure sometimes uses needle blocks (square insertions) that are inserted into the grid fenestrations for needle guidance and for fiducial registration. These insertions are proud of the surface of the grid, and may interfere with the positioning of the gamma camera if they are present when gamma imaging is being done. Therefore, we assume that these insertions can be removed prior to gamma imaging.

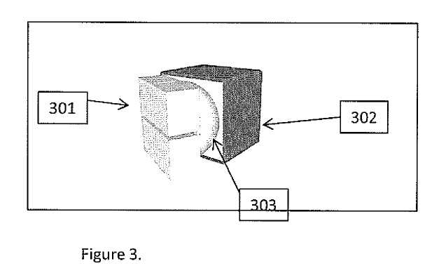

An example of a design for the small gamma camera head is shown in FIG. 3. The small gamma camera head connects in retrofit fashion to the existing square grid immobilization system, and is made of two sections, the CSDE 302 and the square attachment method 301 connected by a swivel joint 303. The front section 301 is designed for mechanical interconnection to the grid and for allowing easy swivel of the camera section. It connects to the NORAS grid using four fenestration inserts and has a dome-shaped swivel joint, which allows for the flexible positioning of the CSDE section. The acronym CSDE stands for collimator, scintillator, detector and electronics, which are the major constituent parts of the scintillation camera which this design represents. Of course, a CZT based direct detection camera could be designed in the same way. Each fenestration of the NORAS square grid has a size of approximately 20×20 mm, and so the gamma camera in this example has a face size of approximately 40×40 mm.

The back section contains the collimator, scintillator and detector and electronics. The front portion is made of plastic or suitable material so that it does not interfere with the gamma camera imaging. The back section is made of plastic with lead lining, to ensure that noise levels within the scintillator are suitable. The cabling (not shown) off of the bottom of the back section of the camera can be clipped to the NORAS mounting post for mechanical support. This mechanical support is important, as the torque on the rear portion of the gamma camera can cause misalignment if there is too much force. A camera of this size weighs approximately 2 pounds. The rear section can be swivelled over at least a 20 to 25 degree swing to allow the operator to position the camera such that the lesion can be properly viewed. The reason for the flexibility in positioning is both to allow SPECT-type imaging to occur as well as to allow the gamma camera to be moved to the side later on so that the biopsy system can be used as well.

The collimator used within this small camera can come in various designs. One suitable design comprises a parallel hole collimator while another suitable version comprises a focusing collimator that offers a four-times improvement in the spatial resolution, imaging time, dosage required, or other performance advantage, as discussed above.

As will be readily apparent to one of skill in the art, various other designs for the gamma camera packaging can be possible.

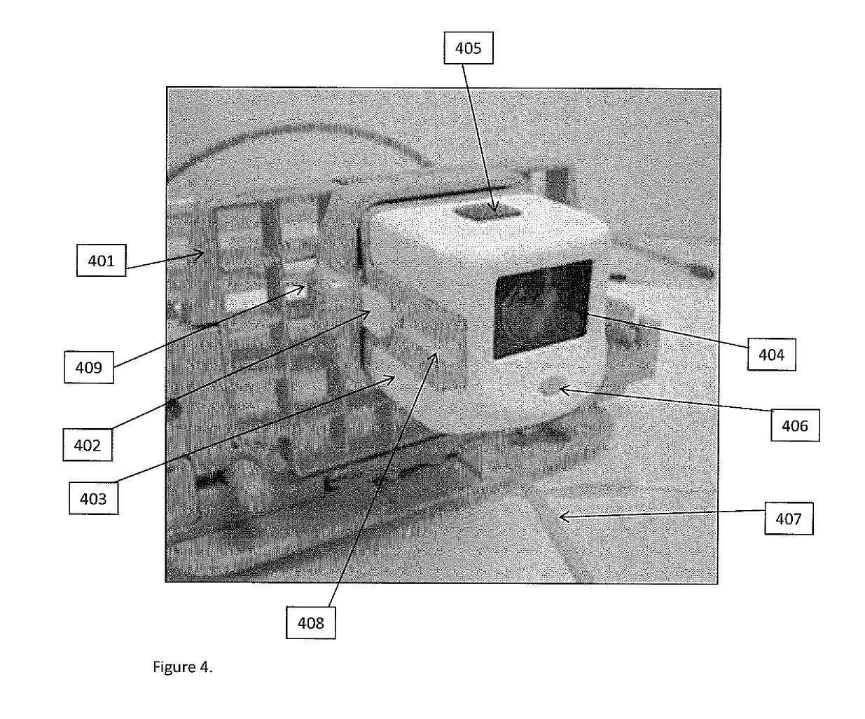

In FIG. 4, a smaller gamma camera head 503 is shown attached to the square grid breast immobilizer 401. There is a female mounting rail 408 on both sides of the gamma camera head that allows a square grid attachment mount 409 to be used. The square grid attachment mount uses a male rail 402 to connect to the gamma camera head. The gamma camera head has a power on button 405 on top, a monitor 404 on the rear face, a display mode switch button 506 below the monitor, and a cable 407 which goes to the interface module. The cable in this case carries power lines of near 30 Vdc, 5Vdc, 2.5Vdc and ground. The detector within the gamma camera head requires the bias voltage near 30 Vdc to operate. Additional lines include the signal lines related to the gamma camera pixels. The size of the gamma camera head can be larger or smaller depending on the details of price, cost, positioning requirements and application requirements. A larger size gamma camera head which fits over the entire breast immobilization grid can also be used in this system.

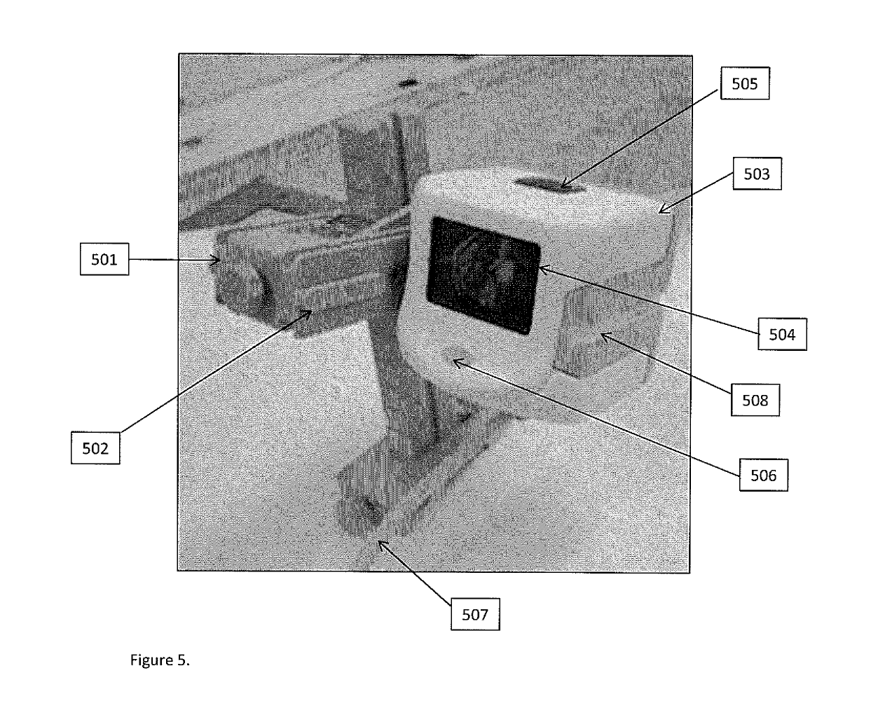

In FIG. 5 is shown an alternative to the square grid system, which is the pillar and post system. One of the limitations of the square grid system is that the biopsy needle must be inserted directly into the breast tissue perpendicular to the breast surface, through a needle block. This means that any lesion that is located directly behind one of the plastic fenestration pieces cannot be targeted. In order to allow some flexibility in targeting the post and pillar system allows the needle to be directed at an upward or downward angle. To attach the small gamma camera head 603 to the pillar and post 501, the male rail 502 of the post is used. The gamma camera head has the female rail 608, and the same design of monitor 504, power button 505, display mode button 506 and cable 507 is present. These post and pillar systems exist in the market today, and so the retrofit design of these camera packages leads to easy usage. In these package designs, an MR safe monitor is designed into the rear of the gamma camera head. This is an option for these systems.

NORAS has built fiducials on the inside of the breast paddle, and these MR fiducials can be used with the positioning accuracy of the equipment to allow mechanical registration of the image sets to be done. This allows an accurate mechanical co-registration to occur, as discussed above.

The NORAS breast immobilization system also swivels about the vertical axis to allow mlo, cc and other orientations of the paddle in relation to the breast. In general, it is desirable to biopsy through as little tissue as possible, and so for those breast lesions that are located on the inside of the breast, it is optimal to rotate the breast paddles and biopsy in the medial direction. For some patient rest systems, the medial direction is challenging because the sternum of the patient rest system dips down, which then decreases the amount of space that the radiologist has in which to biopsy. A small gamma camera is useful in this case, because the gamma imaging can still occur from the medical direction even through space is tight. We assume that the breast paddle position is moved prior to the start of the procedures, and is not moved at all between MR and gamma imaging sessions. For a large gamma camera system, this requirement to allow medial gamma imaging to be possible will limit the maximum size that is allowed.

One of the key advantages of an MR Safe compact Gamma imaging system is the relatively flexible imaging geometries and usages that are possible. The MRI system can be used to identify the lesion locations due to its high level of sensitivity, and then a smaller gamma imaging camera can be used to increase specificity, provide an alternative position and size measurement, provide a functional imaging contribution to the analysis, or be used for real-time biopsy imaging. When the gamma camera size can be reduced, the number of positioning locations that can be used for imaging increases, and this leads to an easier ability to get close to the lesion site and therefore a potential improvement in sensitivity and specificity and a potential improvement in lesion visualization. Also, as the gamma camera size decreases the weight decreases, and therefore this allows one to consider relatively easy movement systems or geometries. A gamma imaging system that uses multiple small gamma camera heads may then be preferable to a system that uses a single large gamma camera head, because the smaller heads have an increased ease of positioning.

### Biopsy Configurations

Using these five configuration items, consisting of larger size gamma camera head, smaller size gamma camera head, monitor, interface module and processing system, and under the assumption that the cabling, analog to digital conversion hardware, signal processing, and presentation software portion of the system is possible through the use of known technologies and methods, it is possible to create various biopsy guidance configurations for the various workflow.

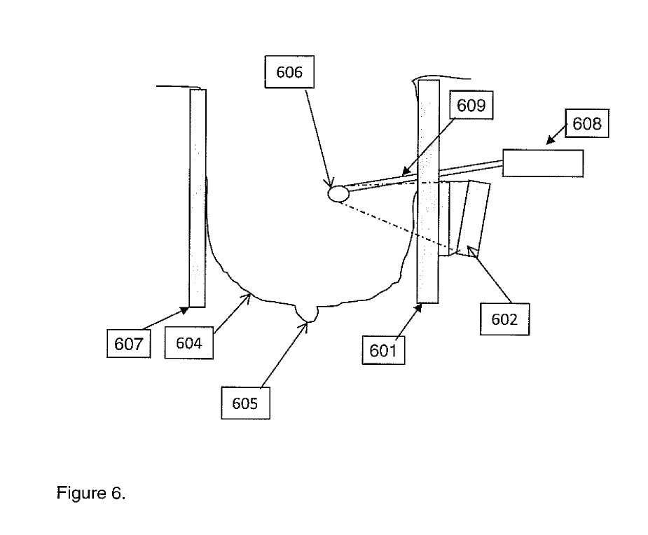

One configuration can have a lead shield in back, the monitor on the side, and a single small camera used on the front paddle, as was shown in FIG. 2. This might be used when a single lesion is being imaged, and the lesion is relatively close to the front paddle. The small camera used might be of the focusing collimator variety, in which case the operator may be checking the spatial outline of the edge of the lesion to see if it qualifies for additional imaging, biopsy workup planning or biopsy guidance. FIG. 6 shows a side view drawing of this approach. In this case, the small gamma camera 602 is mounted onto the square grid breast immobilization system 601, the rear breast paddle 607, and the breast outline 604 and nipple 605 are shown between the paddles. The average width of the held breast will be slightly larger than the compressed breast thickness associated with mammography, and so we expect 8 to 11 cm as a typical breast thickness. The lesion of interest 606 is being targeted by the guide 609 and the biopsy needle system 608. The gamma camera is maintained in position and allows visualization of the lesion during the biopsy process.

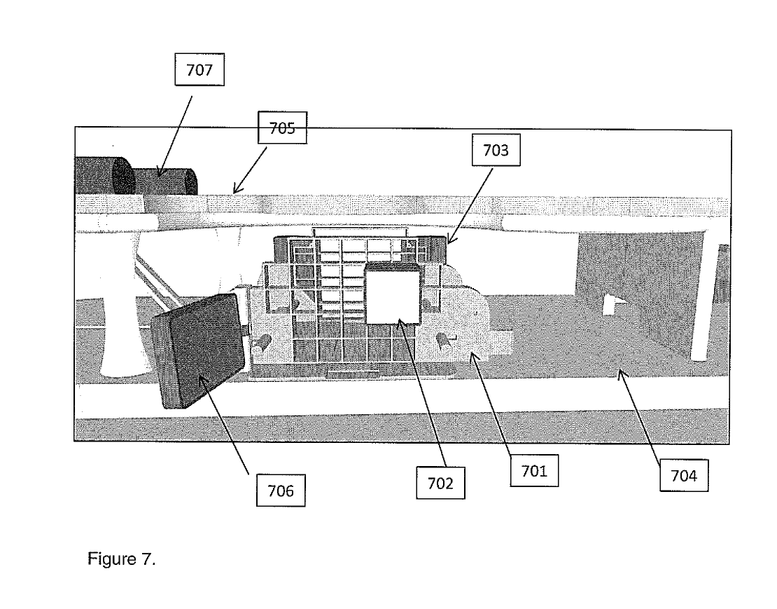

Another configuration shown in FIG. 7 may use a full size gamma camera 703 in the rear and a smaller camera 702 in front, with the front being defined as the side from which biopsy will be done. In this case, the full camera with parallel collimation is obtaining a full view of the breast, and the front small camera may be used to obtain a detailed view of a specific lesion location. The monitor 706 is shown on the left side, the square grid breast immobilization 701 is used and allows the gamma camera to attach to it, and the patient rest system 704, upper padding 705, headrest 707 are all present and are existing equipment pieces in the industry from NORAS and other vendors. This configuration might be used when biopsy guidance is being done, and the full camera in back allows alignment along the bore of the guide and the small camera in front allows alignment of the depth of the guide.

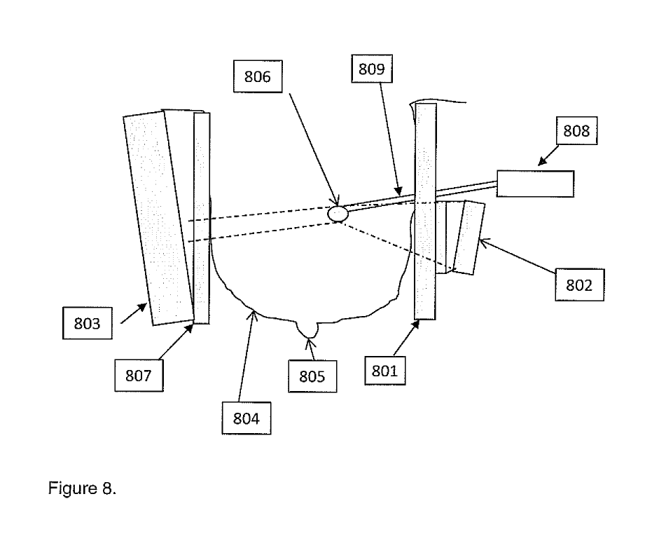

This is shown in FIG. 8, in which the breast outline 804 and nipple 805 are shown between the front breast paddle 801 and the rear breast paddle 807. The small gamma camera head 802 is attached to the front breast paddle using the post and pillar method, the larger gamma camera 803 is attached close to the rear paddle, and the lesion of interest 806 is being targeted by the breast biopsy gun 808 via the biopsy guide 809. There are various types of biopsy guns and guides known in the industry, and the gamma camera is positioned off to one side so that it does not conflict with the existing biopsy equipment. In this case, the rear camera is tilted to allow the rear gamma camera to line up with the guide and needle.

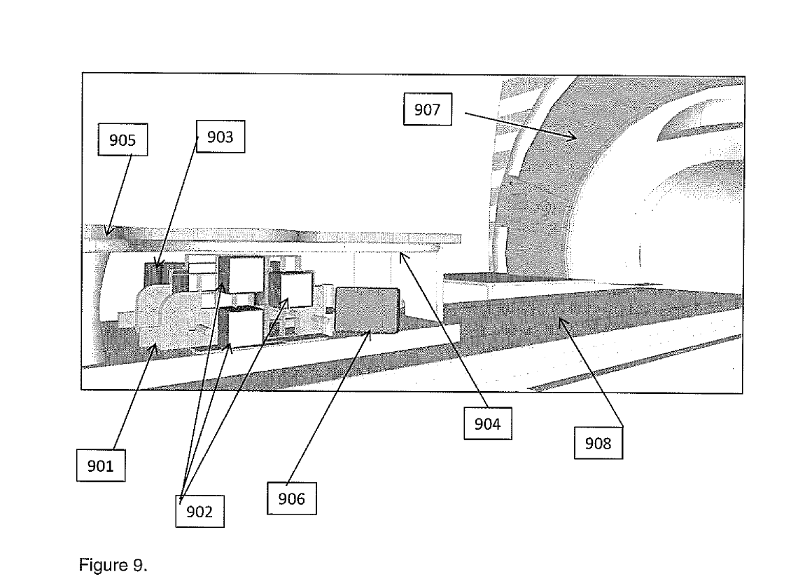

In approximately 2% of MRI breast biopsy cases, there are 3 lesions of interest in the breast. In 14% of cases, there are 2 lesions of interest. One of the methods to approach these multi-lesion cases is to use multiple small gamma cameras, one for each lesion. This allows simultaneous imaging and decreases the time required for the patient to be immobilized in the MRI room. A three camera configuration is shown in FIG. 9. The full camera uses parallel collimation, and the small cameras in front focus in on specific lesion areas.

For all of the above configurations, it is possible to use a second energy to tag the biopsy equipment, and so if the gamma cameras have sufficient energy discrimination they will obtain location information about both lesion and equipment at the same time. This method is known in the art. Because we are using a second energy for the gamma guide, there is very low noise related to this energy. In addition, because the gamma guide will not be in the body too long, there can be relatively high dosage levels inserted into the gamma guide. In addition, because we are assuming that the gamma guide is built in such a way that the radioisotope is not in contact with the patient anatomy, we do not have a risk of the radio-isotope leaking into the patient. All of these reasons allow the use of second radioisotope for the equipment to be advantageous.

In this case of two radioisotopes, the time to acquire the equipment image may be quite short, certainly less than 2 minutes, because this second energy level is quite bright compared to the noise and compared to the 140 keV first energy level associated with the Tc99 that is used for breast imaging.

We will assume that the guide has at least 2 radiospots or radiomarks on it that act as fiducials which must be lined up in order for the guide to point directly at the lesion location. For an 8 cm thick breast, the rear gamma camera will observe the guide as it is inserted into the breast, will observe the at least 2 fiducial marks, and will allow the biopsy system or unit to line up with the lesion location.

The front gamma camera is used to ensure that the guide is not inserted too far. This front gamma camera also has dual energy discrimination ability, and images both the lesion and the guide. At least one fiducial on the guide is positioned at the front tip of the guide, so that the single gamma camera on the front face can identify when the guide is inserted far enough into the patient breast. Our testing within a MR breast phantom indicates that for a 16 pixel gamma camera at a distance of 5 cm from a 0.6 mCi seed of size 1 mm, the counts per second in a pixel can be as high as 50 to 100. This high number of counts means that slow movement of the needle and guide can be tracked by the gamma camera system, allowing real-time biopsy to be obtained.

The small gamma camera, which could be nominally 15×15 mm, 30×30 mm or 45×45 mm in detection area, can be used with a straight through collimator or with a focusing collimator. In this system, the MRI images that are taken in advance of the gamma image session allow the operator to know in advance the location of the lesion in x, y, z coordinates, and therefore the camera with the focusing collimator may be used to achieve higher spatial resolution.

The purpose of these configurations is to obtain improved breast biopsy procedures using the advantages of both MRI and gamma imaging.

While the preferred embodiments of the invention have been described above, it will be recognized and understood that various modifications may be made therein, and the appended claims are intended to cover all such modifications which may fall within the spirit and scope of the invention.

### REFERENCES

[1] “Clinical Experience with MRI-Guided Vacuum-Assisted Breast Biopsy”, Lehman et al, 2005
[2] “Fast MRI-Guided Vacuum Assisted Breast Biopsy” Liberman et al, 2003
[3] “Factors that Impact the Duration of MRI-Guided Core Needle Biopsy”, Norozian et al 2010
[4] “Gamma Guided Stereotactic Breast Biopsy System”, Welch et al, IEEE Trans. On Nuclear Science, vol 53(5), October 2006, pp. 2690-2697.
[5] “Stereotactic Coordinates from ECT Sinograms for radionuclide-guided biopsy”, Raylma, Ficaro and Wahl, J. Nucl. Med. Vol. 37, pp 1562-1567, 1996
[6] “Radionuclied-guided stereotactic prebiopsy localization of nonpalpable breast lesions with normal mammogram”, Khalkhalki, Mishking, Diggles and Klein, J. Nucl. Med. Vol 38 pp 1019-1022, 1997
[7] “Simulated performance of radiotracer guided breast biopsy”, Radiology, vol. 217 p 706, 2000 Kim, Khalkhali, Diggles, Peter, Vargas and Wienberg
[8] “Positron emission mammography-guided breast biopsy”, Raylman, Majewski, Weisenberger, Popov, Wojcik, Kross, Schreiman and Bishop, J. Nude. Med. Vo142, pp. 960-966, 2001
[9] Wienberg, Setpanov, Beylin, Zawazin, Yarnall, Anashkin, Lauckner, Doss and Adler, “Biopsy-ready pem scanner with real-time X-ray correlation capability”, in 2003 IEEE Medical Imaging Conf. Rec. October 2003

1. A method of performing a biopsy comprising:
immobilizing a breast of a patient in need of a biopsy with a breast immobilization apparatus;
inserting the patient into a bore of a magnetic resonance imaging (MRI) device;
taking an MRI image of the breast;
applying a suitable radioisotope for gamma imaging to the patient;
identifying suspicious lesion(s) of interest and their location(s) using the MRI image;
attaching a gamma camera to the breast immobilization apparatus;
visualizing the lesions of interest using the gamma camera; and
performing a biopsy on the selected lesion of interest.

2. The method according to claim 1 wherein the biopsy is performed with a gamma-visible needle.

3. The method according to claim 1 wherein the lesion is imaged during the biopsy with the gamma camera.

4. The method according to claim 1 wherein the gamma camera is used to confirm that biopsy cores are radioactive following the biopsy.

5. The method according to claim 1 wherein the gamma camera is used to confirm that biopsy cores extend from one end of the lesion to the other following the biopsy.

6. The method according to claim 1 wherein the gamma camera is used to determine the radioactive profile of the biopsy cores.

7. The method according to claim 6 wherein the gamma camera is used to confirm that biopsy cores have a radioactive profile in which the tip is not as radioactive as the middle, and in which the ratio of amount of radioactivity in the middle of the core to the amount of radioactivity that is present in the tip of the core can be expressed as a ratio.

8. The method according to claim 1 including two or more gamma cameras.

9. The method according to claim 8 wherein the gamma cameras comprise a first gamma camera having a first scintillator suitable for a first radioisotope and a second gamma camera having a second scintillator suitable for a second radioisotope.

10. The method according to claim 7 wherein the gamma cameras comprise a first gamma camera having a first collimator focus length and a second gamma camera having a second collimator focus length.
